(19) United States
(12) Patent Application Publication
(10) Pub. No.: US 2014/0051744 A1
(43) Pub. Date: Feb. 20, 2014

(54) Markers For Assessing the Susceptibility of Cancer to Survivin-Targeting miRNA Treatment

(71) Applicant: The Regents of the University of Colorado, A Body Corporate, Denver, CO (US)

(72) Inventors: Bolin LIU, Centennial, CO (US); Shuiliang WANG, Fujian (CN)

(73) Assignee: The Regents of the University of Colorado, A Body Corporate, Denver, CO (US)

(21) Appl. No.: 13/966,860

(22) Filed: Aug. 14, 2013

Related U.S. Application Data
(60) Provisional application No. 61/683,470, filed on Aug. 15, 2012.

(51) Int. Cl. A61K 31/7088; A61K 45/06

(52) U.S. Cl. CPC A61K 31/7088; A61K 45/06
USPC 514/44 A; 435/375

(57) ABSTRACT

The present invention relates to the use of miRNA treatment of survivin-positive cancer cells to increase sensitivity of cells to chemotherapy.

### BACKGROUND OF THE INVENTION

This application claims benefit of priority to U.S. Provisional Application Ser. No. 61/683,470, filed Aug. 15, 2012, the entire contents of which are hereby incorporated by reference.

I. Field of the Invention

The present invention relates generally to the fields of oncology and cancer therapy. More particularly, it concerns the assessment of biomarkers that predict the efficacy of an anti-cancer therapy.

II. Description of Related Art

Survivin is the smallest member of IAP (inhibitor of apoptosis) family. It is a dual functional protein acting as an important apoptosis inhibitor and key cell cycle regulator (Liu et al., 2008; Mita et al., 2008). Overexpression of Survivin is frequently observed in human malignancies and positively correlates with poor prognosis, tumor recurrence, and therapeutic resistance (Liu et al., 2008; Mita et al., 2008). Several strategies targeting Survivin have been proposed for cancer therapy (Mita et al., 2008; Altieri, 2008). Among them, immunotherapy, antisense oligonucleotide, and transcriptional suppressors, such as YM155 are actively under preclinical and clinic investigations (Arora et al., 2012; Kelly et al., 2011). YM155 is one of the most advanced studied therapeutics targeting Survivin; and it is currently in multiple clinical trials of human cancers. To date, there is no FDA-approved Survivin-targeted therapy for cancer treatment.

MicroRNAs (miRNAs) are endogenous, small non-coding RNAs that regulate gene expression by targeting mRNAs for degradation or translational repression via sequence-specific recognition (Bartel, 2009; Winter et al., 2009). Recent studies demonstrate that miRNAs can function as oncogenes or tumor suppressors to regulate tumor cell proliferation and apoptosis process, and play highly diverse roles in the development of human cancers (Esquela-Kerscher and Slack, 2006; Nelson and Weiss, 2008; Zhang et al., 2007), including breast cancers (Adams et al., 2008). The inventor previously reported that activation of the erbB3/PI-3K/Akt signaling specifically increased Survivin to confer paclitaxel resistance in erbB2-driven (erbB2+) breast cancer cells (Wang et al., 2010). Subsequent studies on the underlying mechanism suggest erbB3 signaling-mediated upregulation of Survivin is via specific downregulation of two miRNAs, miR-203 and miR-542-3p, which have been shown to target Survivin mRNA (Saini et al., 2011; Yoon et al., 2010). Interestingly, both miR-203 and miR-542-3p have been identified as tumor suppressive miRNAs, and are frequently downregulated in human cancers via promoter methylation (Bray et al., 2011; Craig et al., 2011; Formosa et al., 2012; Furuta et al., 2010; Zhang et al., 2011).

### SUMMARY OF THE INVENTION

Thus, in accordance with the present invention, there is provided a method of inhibiting a survivin-positive cancer cell comprising contacting the cell with miR-203 and/or miR-542-3p in combination with an anti-cancer chemotherapeutic agent. The cancer cell may be contacted with miR-203 and not miR-542-3p, with miR-542-3p and not miR-203, or with miR-203 and miR-542-3p. The anti-cancer chemotherapeutic agent may be an anti-mitotic agent, alkylating agent, and cytotoxic antibiotic. The anti-mitotic agent may be docetaxel, etoposide (VP16), paclitaxel, taxol, taxotere, vinblastine, vincristine, vinorelbine, and vindesine. The alkylating agent may be cisplatin, carboplatin, oxaliplatin, mechlorethamine, cyclophosphamide, chlorambucil, and ifosfamide. The cytotoxic antibiotic may be actinomycin, doxorubicin (adriamycin), valrubicin (valstar), epirubicin (ellence), plicamycin (mithracin), mitomycin C, or bleomycin.

The cancer cell may be a breast cancer cell, prostate cancer cell, ovarian cancer cell, lung cancer cell, gastric cancer cell, esophageal cancer cell, and pancreatic cancer cell. The cancer cell may be a recurrent cancer cell, a metastatic cancer cell or a multi-drug resistant cancer cell. The cancer cell may be contacted with miR-203 and/or miR-542-3p multiple times. The cancer cell may be contacted with the anti-cancer chemotherapeutic agent multiple times. The cancer cell may be contacted with miR-203 and/or miR-542-3p and with the anti-cancer chemotherapeutic agent multiple times. Inhibiting may comprise inhibiting growth of the cancer cell, or may comprise killing of the cancer cell. The method may further comprise contacting the cancer cell with an additional anti-cancer therapy, such as a chemotherapy, a radiotherapy, an immunotherapy, a hormonal therapy or a toxin therapy.

In another embodiment, there is provided a method of treating a subject exhibiting a survivin-positive cancer comprising administering to the subject miR-203 and/or miR-542-3p in combination with an anti-cancer chemotherapeutic agent. The subject may be administered miR-203 and not miR-542-3p, or administered miR-542-3p and not miR-203, or administered miR-203 and miR-542-3p. The anti-cancer chemotherapeutic agent may be an anti-mitotic agent, alkylating agent, and cytotoxic antibiotic. The anti-mitotic agent may be docetaxel, etoposide (VP16), paclitaxel, taxol, taxotere, vinblastine, vincristine, vinorelbine, and vindesine. The alkylating agent may be cisplatin, carboplatin, oxaliplatin, mechlorethamine, cyclophosphamide, chlorambucil, and ifosfamide. The cytotoxic antibiotic may be actinomycin, doxorubicin (adriamycin), valrubicin (valstar), epirubicin (ellence), plicamycin (mithracin), mitomycin C, and bleomycin.

The cancer may be breast cancer, prostate cancer, ovarian cancer, lung cancer, gastric cancer, esophageal cancer, or pancreatic cancer. The cancer may be a recurrent cancer, a metastatic cancer or a multi-drug resistant cancer. The subject may be administered miR-203 and/or miR-542-3p multiple times, may be administered the anti-cancer chemotherapeutic agent multiple times, or may be administered miR-203 and/or miR-542-3p and the anti-cancer chemotherapeutic agent multiple times. Inhibiting may comprise slowing tumor growth, delaying tumor metastasis, or killing one or more cells of the cancer. The method may further comprise treating the subject with an additional anti-cancer therapy, such as a chemotherapy, a radiotherapy, an immunotherapy, a hormonal therapy, a toxin therapy or surgery. The subject may be a non-human mammal or a human. The method may further comprise assessing expression of survivin in a cancer cell from the subject.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### DETAILED DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

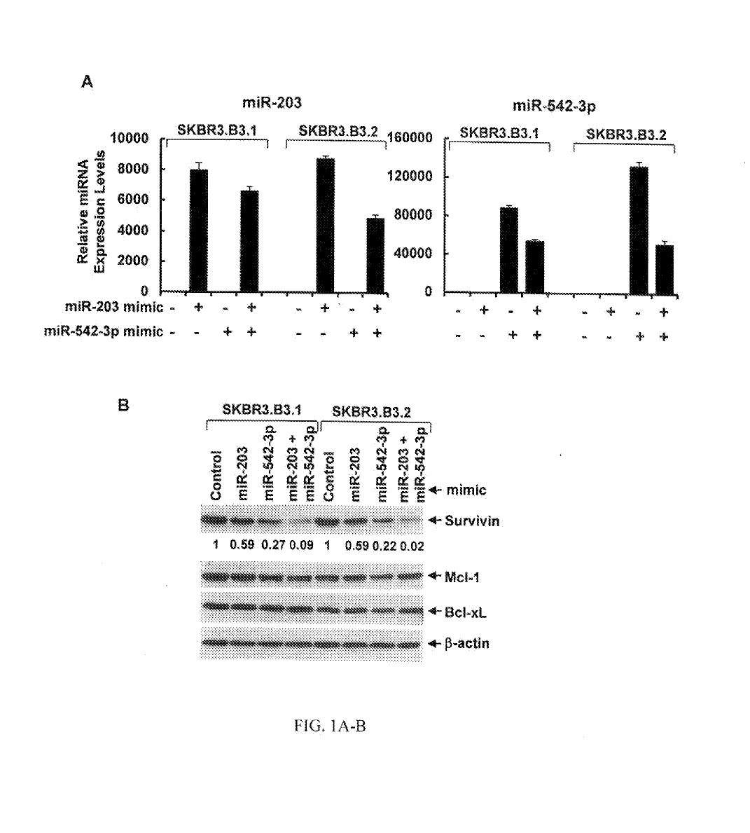

FIGS. 1A-B. Transfection of the mimics of miR-203 and/or miR-542-3p dramatically increases their expression levels and specifically down-regulates Survivin, but not other functionally related molecules, such as Mcl-1 and Bcl-xL. SKBR3.B3.1 and SKBR3.B3.1 cells were transfected with either control miRNA mimic, or miR-203 mimic, or miR-542-3p mimic alone, or the combination of miR-203 and miR-542-3p mimics for 24 hrs. (FIG. 1A) Cells were collected and subjected to total RNA extraction, inclusive of the small RNA fraction. The expression levels of miR-203 and miR-542-3p were measured by qRT-PCR using TaqMan miRNA assays. All results were normalized with the internal control RNU6B. Bars, SD. Data shows the representative of three independent experiments. (FIG. 1B) Cells were collected and subjected to western blot analyses with specific antibodies directed against Survivin, Mcl-1, Bcl-xL, or β-actin. The densitometry analyses of Survivin signals were shown underneath, and the arbitrary numbers indicate the intensities of each cell line relative to controls, defined as 1.0.

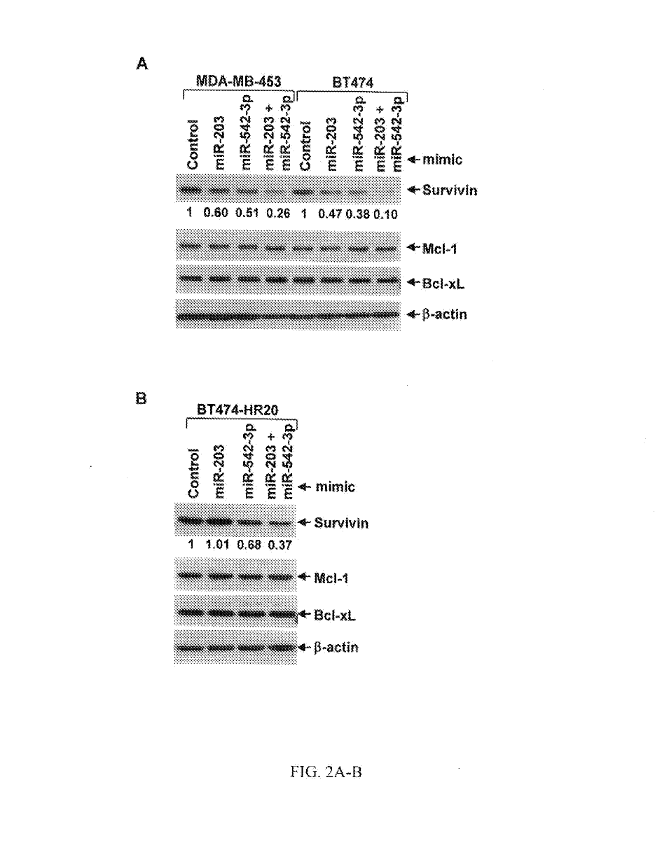

FIGS. 2A-B. MiR-203 and miR-542-3p exhibit functional cooperation to specifically inhibit Survivin in erbB2+ breast cancer cells. MDA-MB-453 and BT474 cells (FIG. 2A), and BT474-HR20 cells (FIG. 2B) were transfected with either control miRNA mimic, or miR-203 mimic, or miR-542-3p mimic alone, or the combination of miR-203 and miR-542-3p mimics for 24 hrs. Cells were then collected and subjected to western blot analyses with specific antibodies directed against Survivin, Mcl-1, Bcl-xL, or β-actin. The densitometry analyses of Survivin signals were shown underneath, and the arbitrary numbers indicate the intensities of each cell line relative to controls, defined as 1.0.

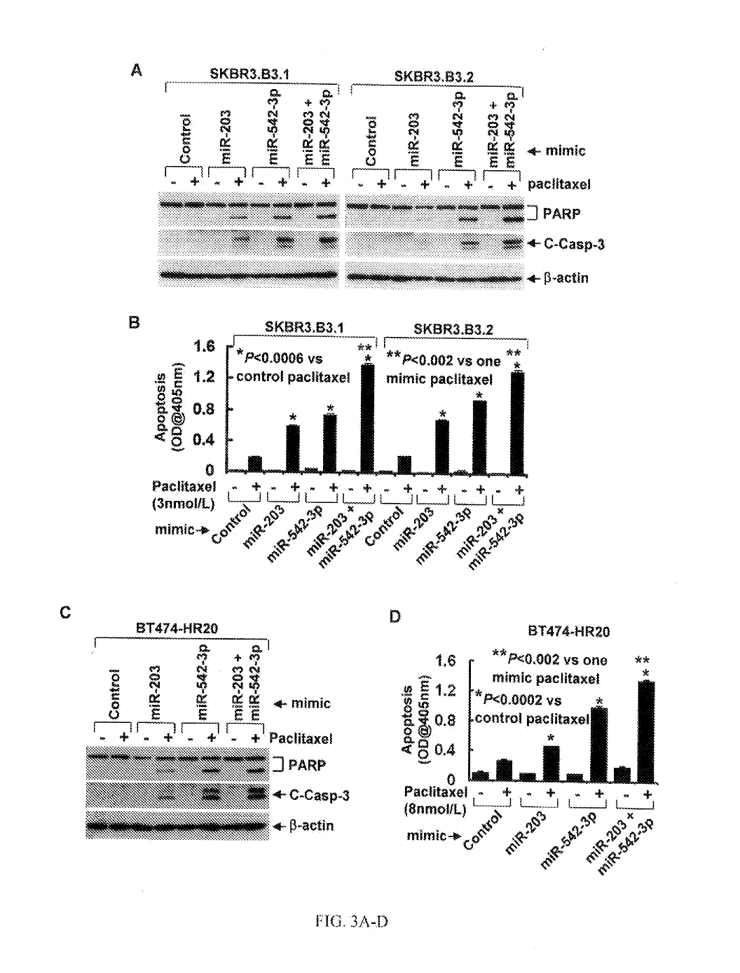

FIGS. 3A-D. It appears miR-203 and miR-542-3p act in concert to significantly enhance paclitaxel-induced apoptosis in the otherwise resistant breast cancer cells. (FIGS. 3A-B) SKBR3.B3.1 and SKBR3.B3.2 cells were transfected with either control miRNA mimic, or miR-203 mimic, or miR-542-3p mimic alone, or the combination of miR-203 and miR-542-3p mimics for 24 hrs. Cells were then untreated or treated with paclitaxel for another 24 hrs. Cells were collected and subjected to western blot analyses of PARP, cleaved caspase-3 (C-Caps-3), β-actin (FIG. 3A), or a specific apoptotic ELISA (FIG. 3B). C & D, BT474-HR20 cells were transfected with either control miRNA mimic, or miR-203 mimic, or miR-542-3p mimic alone, or the combination of miR-203 and miR-542-3p mimics for 24 hrs. Cells were then untreated or treated with paclitaxel for another 24 hrs. Cells were collected and subjected to western blot analyses of PARP, cleaved caspase-3 (C-Caps-3), or β-actin (FIG. 3C), or a specific apoptotic ELISA (FIG. 3D).

### DESCRIPTION OF THE ILLUSTRATIVE EMBODIMENTS

As discussed above, the that activation of the erbB3/PI-3K/Akt signaling pathways has been shown to specifically increase Survivin expression and confer paclitaxel resistance in erbB2-driven (erbB2+) breast cancer cells (Wang et al., 2010). Subsequent studies also indicated that erbB3 signaling-mediated upregulation of Survivin occurs via downregulation of miR-203 and miR-542-3p, which have been shown to target Survivin mRNA (Saini et al., 2011; Yoon et al., 2010). Interestingly, both miR-203 and miR-542-3p have been identified as tumor suppressive miRNAs, and are frequently downregulated in human cancers via promoter methylation (Bray et al., 2011; Craig et al., 2011; Formosa et al., 2012; Furuta et al., 2010;

Zhang et al., 2011). Thus, the inventor hypothesized that reactivation of miR-203 and/or miR-542-3p via exogenous expression may potently inhibit Survivin, and therefore significantly enhance the efficacy of chemotherapeutic agents for cancer treatment.

To test this hypothesis, the inventor took advantage of the paclitaxel-resistant breast cancer cells-established in the inventor's laboratory (Wang et al., 2010; Huang et al., 2010). Having studied the role of ectopic expression of miR-203 and/or miR-542-3p in regulating Survivin expression, and in enhancing paclitaxel-induced apoptosis in the otherwise resistant breast cancer cells, the new data presented here suggest that reactivation of the Survivin-targeting miRNAs may be efficacious to potentiate cancer chemotherapy. These and other aspects of the invention are discussed in detail below.

### I. Cancer and Targeted Treatments

A. Cancers

Exemplary cancers for which treatment is contemplated in the present invention include lung cancer (including non-small cell lung cancer), esophageal cancer, prostate cancer, pancreatic cancer, gastric cancer, ovarian cancer, and breast cancer.

The cancer may be treated after its initial diagnosis or subsequently by therapies or combination of two or more therapies. A cancer recurrence may be defined as the reappearance or rediagnosis of a patient as having any cancer following any treatment including one or more of surgery, radiotherapy or chemotherapy. The patient with relapsed disease need not have been reported as disease free, but merely that the patient has exhibited renewed cancer growth following some degree of clinical response by the first therapy.

The therapy or clinical response may result in, but is not limited to, stable disease, tumor regression, tumor necrosis, absence of demonstrable cancer, reduction in tumor size or burden, blocking of tumor growth, reduction in tumor-associated pain, reduction in tumor associated pathology, reduction in tumor associated symptoms, tumor non-progression, increased disease free interval, increased time to progression, induction of remission, reduction of metastasis, or increased patient survival.

B. ErbB-2

The epidermal growth factor receptor (EGFR) is the founding member of the ErbB family of four structurally-related receptor tyrosine kinases. In humans, this includes Her1 (EGFR, ErbB1), Her2 (Neu, ErbB2), Her3 (ErbB3), and Her4 (ErbB4). The gene symbol, ErbB, is derived from the name of a viral oncogene to which these receptors are homologous: Erythroblastic Leukemia Viral Oncogene. Insufficient ErbB signaling in humans is associated with the development of neurodegenerative diseases, such as multiple sclerosis and Alzheimer's Disease. In mice loss of signaling by any member of the ErbB family results in embryonic lethality with defects in organs including the lungs, skin, heart, and brain. Excessive ErbB signaling is associated with the development of a wide variety of types of solid tumor. ErbB-1 and ErbB-2 are found in many human cancers, and their excessive signaling may be critical factors in the development and malignancy of these tumors.

The ErbB protein family consists of 4 members: ErbB-1, also named epidermal growth factor receptor (EGFR), ErbB-2, also named HER2 in humans and neu in rodents, ErbB-3, also named HER3 and ErbB-4, also named HER4. v-ErbBs are homologous to EGFR, but lack sequences within the ligand binding ectodomain. These four members are capable of forming homodimers, heterodimers, and possibly higher-order oligomers upon activation by a subset of potential growth factor ligands. There are 11 growth factors that activate ErbB receptors.

When not bound to a ligand the extracellular regions of ErbB-1, -3, and -4 are found in a ‘tethered’ conformation in which a 10-amino-acid-long dimerization arm is unable to mediate monomer-monomer interactions. In contrast, in ligand-bound ErbB-1 and unliganded ErbB-2, the dimerization arm becomes untethered and exposed at the receptor surface, making monomer-monomer interactions and dimerization possible. The consequence of ectodomain dimerization is the positioning of two cytoplasmic domain such that transphosphorylation of specific tyrosine, serine, and threonine amino acids can occur within the cytoplasmic domain of each ErbB. At least 10 specific tyrosines, 7 serines, and 2 threonines have been identified within the cytoplasmic domain of ErbB-1, that may become phosphorylated and in some cases de-phosphorylated (e.g., Tyr 992) upon receptor dimerization. Although a number of potential phosphorylation sites exist, upon dimerization only one or much more rarely two of these sites are phosphorylated at any one time.

### II. Survivin

Survivin, also called Baculoviral IAP repeat-containing 5 (BIRC5), is a human gene that is part of the inhibitor of apoptosis family (IAP). The survivin protein functions to inhibit caspase activation therefore leading to negative regulation of apoptosis or programmed cell death. This has been shown by disruption of survivin induction pathways leading to increase in apoptosis and decrease in tumor growth. The survivin protein is expressed highly in most human tumors and fetal tissue, but is completely absent in terminally differentiated cells. This fact therefore makes survivin an ideal target for cancer therapy as cancer cells are targeted while normal cells are left alone. Survivin expression is also highly regulated by the cell cycle and is only expressed in the G2-M phase. It is known that survivin localizes to the mitotic spindle by interaction with tubulin during mitosis and may play a contributing role in regulating mitosis. The molecular mechanisms of survivin regulation are still not well understood to this day, but regulation of survivin seems to be linked to the p53 protein.

A structural feature common to all IAP family proteins is that they all contain at least one baculoviral IAP repeat (BIR) domain characterized by a conserved zinc-coordinating Cys/His motif at the N-terminal half of the protein. Survivin is distinguished from other IAP family members in that it has only one BIR domain. The mice and human BIR domain of survivin are very similar structurally except for two differences which may affect function variability. The human survivin also contains an elongated C-terminal helix comprising 42 amino acids. Survivin is 16.5 kDa large and is the smallest member of the IAP family. The accession number for the mRNA is NM 001012270.

Thus, in accordance with the present invention, certain cancers may be classified as “surviving-expressing cancers,” which exhibit detectable survivin expression as measured by standard techniques such as immunohistochemistry.

III. miRNAs

A. Background

In 2001, several groups used a novel cloning method to isolate and identify a large group of “microRNAs” (miRNAs) from C. elegans, Drosophila, and humans (Lagos-Quintana et al., 2001; Lau et al., 2001; Lee and Ambros, 2001). Several hundreds of miRNAs have been identified in plants and animals—including humans—which do not appear to have endogenous siRNAs. Thus, while similar to siRNAs, miRNAs are nonetheless distinct.

miRNAs thus far observed have been approximately 21-22 nucleotides in length and they arise from longer precursors, which are transcribed from non-protein-encoding genes. See review of Carrington et al. (2003). The precursors form structures that fold back on each other in self-complementary regions; they are then processed by the nuclease Dicer in animals or DCL1 in plants. miRNA molecules interrupt translation through precise or imprecise base-pairing with their targets.

miRNAs are transcribed by RNA polymerase II and can be derived from individual miRNA genes, from introns of protein coding genes, or from poly-cistronic transcripts that often encode multiple, closely related miRNAs. Pre-miRNAs, generally several thousand bases long are processed in the nucleus by the RNase Drosha into 70- to 100-nt hairpin-shaped precursors. Following transport to the cytoplasm, the hairpin is further processed by Dicer to produce a double-stranded miRNA. The mature miRNA strand is then incorporated into the RNA-induced silencing complex (RISC), where it associates with its target mRNAs by base-pair complementarity. In the relatively rare cases in which a miRNA base pairs perfectly with an mRNA target, it promotes mRNA degradation. More commonly, miRNAs form imperfect heteroduplexes with target mRNAs, affecting either mRNA stability or inhibiting mRNA translation.

The 5′ portion of a miRNA spanning bases 2-8, termed the ‘seed’ region, is especially important for target recognition (Krenz and Robbins, 2004; Kiriazis and Krania, 2000). The sequence of the seed, together with phylogenetic conservation of the target sequence, forms the basis for many current target prediction models. Although increasingly sophisticated computational approaches to predict miRNAs and their targets are becoming available, target prediction remains a major challenge and requires experimental validation. Ascribing the functions of miRNAs to the regulation of specific mRNA targets is further complicated by the ability of individual miRNAs to base pair with hundreds of potential high and low affinity mRNA targets and by the targeting of multiple miRNAs to individual mRNAs.

The first miRNAs were identified as regulators of developmental timing in C. elegans, suggesting that miRNAs, in general, might play decisive regulatory roles in transitions between different developmental states by switching off specific targets (Fatkin et al., 2000; Lowes et al., 1997). However, subsequent studies suggest that miRNAs, rather than functioning as on-off “switches,” more commonly function to modulate or fine-tune cell phenotypes by repressing expression of proteins that are inappropriate for a particular cell type, or by adjusting protein dosage. miRNAs have also been proposed to provide robustness to cellular phenotypes by eliminating extreme fluctuations in gene expression.

Research on microRNAs is increasing as scientists are beginning to appreciate the broad role that these molecules play in the regulation of eukaryotic gene expression. The two best understood miRNAs, lin-4 and let-7, regulate developmental timing in C. elegans by regulating the translation of a family of key mRNAs (reviewed in Pasquinelli, 2002). Several hundred miRNAs have been identified in C. elegans, Drosophila, mouse, and humans. As would be expected for molecules that regulate gene expression, miRNA levels have been shown to vary between tissues and developmental states. In addition, one study shows a strong correlation between reduced expression of two miRNAs and chronic lymphocytic leukemia, providing a possible link between miRNAs and cancer (Calin, 2002). Although the field is still young, there is speculation that miRNAs could be as important as transcription factors in regulating gene expression in higher eukaryotes.

There are a few examples of miRNAs that play critical roles in cell differentiation, early development, and cellular processes like apoptosis and fat metabolism. lin-4 and let-7 both regulate passage from one larval state to another during C. elegans development (Ambros, 2003). mir-14 and bantam are drosophila miRNAs that regulate cell death, apparently by regulating the expression of genes involved in apoptosis (Brennecke et al., 2003, Xu et al., 2003). miR-14 has also been implicated in fat metabolism (Xu et al., 2003). Lsy-6 and miR-273 are C. elegans miRNAs that regulate asymmetry in chemosensory neurons (Chang et al., 2004). Another animal miRNA that regulates cell differentiation is miR-181, which guides hematopoietic cell differentiation (Chen et al., 2004). These molecules represent the full range of animal miRNAs with known functions. Enhanced understanding of the functions of miRNAs will undoubtedly reveal regulatory networks that contribute to normal development, differentiation, inter- and intracellular communication, cell cycle, angiogenesis, apoptosis, and many other cellular processes. Given their important roles in many biological functions, it is likely that miRNAs will offer important points for therapeutic intervention or diagnostic analysis.

Characterizing the functions of biomolecules like miRNAs often involves introducing the molecules into cells or removing the molecules from cells and measuring the result. If introducing a miRNA into cells results in apoptosis, then the miRNA undoubtedly participates in an apoptotic pathway. Methods for introducing and removing miRNAs from cells have been described. Two recent publications describe antisense molecules that can be used to inhibit the activity of specific miRNAs (Meister et al., 2004; Hutvagner et al., 2004), and others have proven their functionality in the heart, where they efficiently knocked-down miR-133 and miR-1 (Care et al. 2007; Yang et al. 2007). Another publication describes the use of plasmids that are transcribed by endogenous RNA polymerases and yield specific miRNAs when transfected into cells (Zeng et al., 2002). These two reagent sets have been used to evaluate single miRNAs.

B. miR-203

In molecular biology miR-203 is a short non-coding RNA molecule. MicroRNAs function to regulate the expression levels of other genes by several mechanisms, such as translational repression and Argonaute-catalyzed messenger RNA cleavage. miR-203 has been identified as a skin-specific microRNA, and it forms an expression gradient that defines the boundary between proliferative epidermal basal progenitors and terminally differentiating suprabasal cells. It has also been found upregulated in psoriasis and differentially expressed in some types of cancer.

In mice, miR-203 is located in chromosome 12, within a fragile 7-Mb region that is lost is some hematopoietic malignancies. This region encodes 52 mature miRNAs, ˜12% of the mammalian miRNA genome. In humans, this region is conserved and located intergenically in 14q32. This microRNA was predicted using computational tools by comparison to mouse and tiger blowfish sequences. It has been validated in zebrafish and its expression confirmed in humans by cloning and sequencing, where it was found in the outer layer of epidermis.

miR-203 has one validated target, p63, conserved across vertebrate lineages. p63 is an essential regulator of stem cell maintenance in stratified epithelial tissues. There is some controversy as to whether suppressor of cytokine signaling 3 (SOCS3) is also targeted by miR-203. Another putative target is ABL1, which is found activated in hematopoietic malignancies where miR-203 is epigenetically silenced by hypermethylation.

The expression of miR-203 is significantly upregulated between E13.5 and E15.5 day mice, suggesting that it may be absent from multipotent progenitors of single-layered epidermis, but is induced upon stratification and differentiation. It also was expressed at high levels in differentiating cells such as hair follicles, epidermis and sebaceous glands. miR-203 expression is first detectable at 17 weeks gestation in the suprabasal layers of epidermis, and this localization was conserved in the adult skin. When miR-203 is expressed prematurely, basal cells diminish their proliferative potential; and when it is absent, proliferation is no longer restricted to the basal layer of epidermis.

miR-203 has been found overexpressed in pancreatic adenocarcinoma and shows correlation with poor prognosis in patients that had undergone pancreatectomy, and has been suggested as a new prognostic marker for this disease. Also, miR-203 has been identified as target of human papillomavirus (HPV) protein E7, which causes its downregulation and thus de-repression of p63 and its downstream targets, ensuring proliferative potential on the host cell, required for the virus to replicate. High levels of miR-203 are inhibitory of HPV amplification.

miR-203 has also been proposed as a tumor-suppressive microRNA in hepatocellular carcinoma (HCC) and hematopoietic malignancies. miR-203, along with miR-124, is epigenetically silenced in primary HCC tumors compared with non-tumorous liver tissues. Also, expression of miR-203 in HCC cells lacking their expression inhibited cell growth and downregulated a set of other possible targets. Others have found silencing of miR-203 in some leukemias, as well as an inverse correlation between miR-203 and ABL1 levels (sometimes expressed as the BCR-ABL1 fusion protein). Supporting its role as a tumor suppressor, it has also been found upregulated upon UVC irradiation in the squamous cell carcinoma lines, suggesting a connection between miR-203 and the activation of the apoptotic program.

miR-203, along with miR-146a, miR-21, and miR-125b, has been identified as a psoriasis-specific microRNA when compared with healthy human skin or atopic eczema. They also observed downregulation of SOCS3 concurrently with upregulation of miR-203 in psoriatic plaques, potentially having an effect in inflammatory responses. Overexpression of miR-203 in rheumatoid arthritis synovial fibroblasts (RASFs) compared to healthy or osteoarthritis samples; and enforced expression of miR-203 led to higher levels of MMP-1 and IL-6 and thus contributed to the activated phenotype of RASFs. MiR-203 regulation was found to be methylation-dependent.

The miR-203 sequence is shown below:

GUGAAAUGUUUAGGACCACUAG

The accession number for human miR-203 is MIMAT0000264.

C. miR-542-3p

miR-542-3p is one of the mature forms of miR-542, whose gene locates at Xq26.3 in human. The other mature form of miR-542 is miR-542-5p.

Studies on miR-542-3p in cancer research are limited. A recent report with microarray analysis (Yoon et al., 2010) revealed that the most down-regulated candidate gene was Survivin in cells transfected with miR-542-3p mimics, as compared with negative control-transfected cells. MiR-542-3p may down-regulate endogenous Survivin at its mRNA and/or protein levels.

As predicted by the miRNA database, like TargetScan, there are three conserved binding sites for miR-542-3p in the 3′-UTR of Survivin. Yoon et al. (2010) found that miR-542-3p directly regulated the expression of Survivin mainly through its interaction with the predicted binding site 2 in Survivin's 3′-UTR. Ectopic expression of miR-542-3p inhibited cancer cell proliferation through G1 and G2/M cell cycle arrest. Additionally, overexpression of miR-542-3p significantly decreased 18rRNA levels, which could affect the process of oncogenic protein synthesis. Collectively, this study demonstrates that Survivin is a direct target of miR-542-3p and growth inhibition by miR-542-3p may have a potential utility as an anti-cancer therapy. The non-receptor tyrosine kinase Src is upregulated in various human cancers. Oneyama et al. (2012) recently reported that downregulation of miR-542-3p is tightly associated with tumor progression via Src-associated oncogenic pathways. In Src-transformed fibroblasts and human cancer cells, miR-542-3p is substantially downregulated, and the ectopic expression of miR-542-3p suppresses tumor growth. The integrin-linked kinase (ILK) has been identified as a conserved target of miR-542-3p. ILK upregulation promotes cell adhesion and invasion by activating the integrin-focal adhesion kinase (FAK)/Src pathway, and can also contribute to tumor growth via the Akt and glycogen synthase kinase 3β pathway. The expression of miR-542-3p is downregulated by the activation of Src-related signaling molecules, such as the epidermal growth factor receptor (EGFR), K-Ras, and Ras/Raf/mitogen-activated protein kinase (MAPK). In human colon cancer tissues, downregulation of miR-542-3p is significantly correlated with the upregulation of Src and ILK. These results suggest that the Src-miR-542-3p-ILK-FAK circuit plays a crucial role in controlling tumor progression.

The miR-542-3p sequence is shown below:

UGUGACAGAUUGAUAACUGAAA

The accession number for human miR-542-3p is MIMAT0003389.

D. Agonists of miR-203 and miR-542-3p

Agonists of miRNAs will generally take one of three forms. First, there is miRNA itself. Such molecules may be delivered to target cells, for example, by injection or infusion, optionally in a delivery vehicle such as a lipid, such as a liposome or lipid emulsion. Second, one may use expression vectors that drive the expression of the miRNA. The composition and construction of various expression vectors is described elsewhere in the document. Third, one may use agents distinct from miRNA that act up-regulate, stabilize or otherwise enhance the activity of miR, including small molecules. Such molecules include “mimetics,” molecules which mimic the function, and possibly form of miRNA, but are distinct in chemical structure.

A wide variety of well-known, alternative oligonucleotide chemistries may be used (see, e.g., U.S. Patent Publications 2007/0213292, 2008/0032945, 2007/0287831, etc.), particularly single-stranded complementary oligonucleotides comprising 2′ methoxyethyl, 2′-fluoro, and morpholino bases (see e.g., Summerton and Weller, 1997). The oligonucleotide may include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). Also contemplated are locked nucleic acid (LNA) and peptide nucleic acids (PNA).

IV. Expression Vectors Encoding miRNAs

A. Therapeutic Regimens

The present invention contemplates the use of agonists of miRNAs in the treatment of surivin-positive cancers. In general, agonists will be small molecules, expression constructs expressing the miRNAs (discussed below), or the miRNAs themselves. Any of these agents may be delivered in a pharmacologically acceptable delivery medium, such as but not limited to, lipid moieties such as liposomes, nanoparticles and the like.

Treatment regimens would vary depending on the clinical situation, with earliest intervention being sought. However, treatment for at least some period of time, such as that involving multiple administrations, would appear to be appropriate in some circumstances. It also may be desirable treat with modulators of miRNAs intermittently, or to vary which miRNAs are given, in order to maximize the protective effects. Combinations with known therapies also are contemplated.

An miRNA can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of miRNA (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of miRNA per kg of bodyweight. The unit dose, for example, can be administered by injection (e.g., intravenous or intramuscular, intrathecally, or directly into an organ), inhalation, or a topical application.

Delivery of an miRNA directly to an organ can be at a dosage on the order of about 0.00001 mg to about 3 mg per organ, or particularly about 0.0001-0.001 mg per organ, about 0.03-3.0 mg per organ, about 0.1-3.0 mg per organ or about 0.3-3.0 mg per organ.

Significant effects may be achieved using nanomolar/submicromolar or picomolar/subnamomolar concentrations of the oligonucleotide, and it is typical to use the lowest concentration possible to achieve the desired resultant increased synthesis, e.g., oligonucleotide concentrations in the 1-100 nM range are contemplated; more particularly, the concentration is in the 1-50 nM, 1-25 nM, 1-10 nM, or picomolar range. In particular embodiments, the contacting step is implemented by contacting the cell with a composition consisting essentially of the oligonucleotide.

In one embodiment, the miRNA is delivered as a unit dose that is administered once a day, e.g., or less frequently less than or at about every 2, 4, 8 or 30 days. In another embodiment, the unit dose is not administered with a frequency (e.g., not a regular frequency). For example, the unit dose may be administered a single time. Because oligonucleotide agent can persist for several days after administering, in many instances, it is possible to administer the composition with a frequency of less than once per day, or, for some instances, only once for the entire therapeutic regimen.

B. miRNA Expression

As an alternative to administering the miRNA itself, it is possible to express the miRNA from an expression vector that is provided to the cell or subject. In particular, lentiviral vector systems are commercially available for using in miRNA expression, and have proved safe for human administration.

For example, Cell Biolabs, Inc. (San Diego Calif.) provides ViraSafe™ lentiviral vectors engineered to minimize the chance of making replication-competent lentivirus (RCL). The ViraSafe™ technology goes beyond other 3rd generation systems with sequence homology reduced an additional 80-90%, while still using transfection of four plasmids. The packaging plasmids are provided individually, not premixed, to allow optimization of vector ratios. ViraSafe™ Lentiviral Expression Systems are complete systems that include three packaging vectors, an expression vector, and a control vector

SystemBio (Mountainview Calif.) provides vectors for delivery and permanent miRNA overexpression with their microRNA Lentiviral Cloning and Expression System. One can easily and efficiently transduce dividing and quiescent mammalian cells—including difficult-to-transfect cells such as primary, stem, neuronal, and endothelial. In addition, our lentivectors can be used to generate microRNA overexpression in animals, such as transgenic mice. The Lentivector system provides a convenient and effective approach to create stable cell lines or transgenic animals since it efficiently integrates the microRNA expression construct of your choice into genomic DNA. Assays with cell lines or transgenic animals that permanently and heritably maintain the overexpression phenotype. SBI's HIV and FIV-based Cloning and Expression Lentivectors are derived from self-inactivating lentiviral vectors. This system offers a highly effective and very safe approach for introducing and expressing any microRNA sequence in nearly any mammalian cell system.

Clontech (Mountainview Calif.) offers their Lentiviral Vector Systems with an EF-1 Alpha Promoter. Lenti-X lentiviral vector systems with EF-1 alpha promoters allow you to achieve robust, constitutive, long-term expression of a gene of interest in cell types in which CMV promoters are often silenced, such as hematopoietic and stem cells. The EF-1 alpha promoter, which offers a broad host range, is derived from the human EEF1A1 gene that expresses the alpha subunit of eukaryotic elongation factor 1.

C. Assessing of Survivin Expression

Assessment of expression levels of surviving in cancer cells may be direct, as in the use of quantitative immunohistochemistry (IHC) or other antibody-based assays (Western blot, FIA, FISH, radioimmunoassay (RIA), RIP, ELISA, immunoassay, immunoradiometric assay, a fluoroimmunoassay, an immunoassay, a chemiluminescent assay, a bioluminescent assay, a gel electrophoresis), or indirectly by quantitating the transcripts for these genes (in situ hybridization, nuclease protection, Northern blot or PCR, including RT-PCR). Relevant methodologies are discussed below.
1. Nucleic Acid-Based Methods

The present invention also comprises methods of examining mRNA expression as a measure of target protein levels. mRNA is isolated from cancer cells according to standard methodologies (Sambrook et al., 1989). It may be desired to convert the RNA to a complementary DNA. In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

A variety of different assays are contemplated, including but not limited to, fluorescent in situ hybridization (FISH), Northern blotting, dot blot analysis, and of course PCR and RT-PCR.
i. Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process. In particular embodiments, the probes or primers are labeled with radioactive species (³²P, ¹⁴C, ³⁵S, ³H, or other label), with a fluorophore (rhodamine, fluorescein) or a chemillumiscent (luciferase).
ii. Template Dependent Amplification Methods

A number of template dependent processes are available to amplify the marker sequences present in a given template sample. One of the best known amplification methods is the polymerase chain reaction (referred to as PCR™ ) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al., 1989. Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention, Walker et al., (1992).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR™-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double-stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single-stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR™” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide,” thereby amplifying the di-oligonucleotide, may also be used in the amplification step of the present invention. Wu et al., (1989), incorporated herein by reference in its entirety.

Real-time polymerase chain reaction, also called quantitative real time polymerase chain reaction (qPCR) or kinetic polymerase chain reaction, is a laboratory technique based on the polymerase chain reaction, which is used to amplify and simultaneously quantify a targeted DNA molecule. It enables both detection and quantification (as absolute number of copies or relative amount when normalized to DNA input or additional normalizing genes) of a specific sequence in a DNA sample.

The procedure follows the general principle of polymerase chain reaction; its key feature is that the amplified DNA is quantified as it accumulates in the reaction in real time after each amplification cycle. Two common methods of quantification are the use of fluorescent dyes that intercalate with double-stranded DNA, and modified DNA oligonucleotide probes that fluoresce when hybridized with a complementary DNA.

Frequently, real-time polymerase chain reaction is combined with reverse transcription polymerase chain reaction to quantify low abundance messenger RNA (mRNA), enabling a researcher to quantify relative gene expression at a particular time, or in a particular cell or tissue type. Although real-time quantitative polymerase chain reaction is often marketed as RT-PCR, it should not be confused with reverse transcription polymerase chain reaction, also known as RT-PCR.

A DNA-binding dye binds to all double-stranded (ds)DNA in a PCR reaction, causing fluorescence of the dye. An increase in DNA product during PCR therefore leads to an increase in fluorescence intensity and is measured at each cycle, thus allowing DNA concentrations to be quantified. However, dsDNA dyes such as SYBR Green will bind to all dsDNA PCR products, including non-specific PCR products (such as “primer dimers”). This can potentially interfere with or prevent accurate quantification of the intended target sequence. The reaction is prepared as usual, with the addition of fluorescent dsDNA dye.

The reaction is run in a thermocycler, and after each cycle, the levels of fluorescence are measured with a detector; the dye only fluoresces when bound to the dsDNA (i.e., the PCR product). With reference to a standard dilution, the dsDNA concentration in the PCR can be determined.

Like other real-time PCR methods, the values obtained do not have absolute units associated with it (i.e. mRNA copies/cell). As described above, a comparison of a measured DNA/RNA sample to a standard dilution will only give a fraction or ratio of the sample relative to the standard, allowing only relative comparisons between different tissues or experimental conditions. To ensure accuracy in the quantification, it is usually necessary to normalize expression of a target gene to a stably expressed gene. This can correct possible differences in RNA quantity or quality across experimental samples.

Using fluorescent reporter probes is the most accurate and most reliable of the methods, but also the most expensive. It uses a sequence-specific RNA or DNA-based probe to quantify only the DNA containing the probe sequence; therefore, use of the reporter probe significantly increases specificity, and allows quantification even in the presence of some non-specific DNA amplification. This potentially allows for multiplexing—assaying for several genes in the same reaction by using specific probes with different-colored labels, provided that all genes are amplified with similar efficiency.

It is commonly carried out with an RNA-based probe with a fluorescent reporter at one end and a quencher of fluorescence at the opposite end of the probe. The close proximity of the reporter to the quencher prevents detection of its fluorescence; breakdown of the probe by the 5′ to 3′ exonuclease activity of the taq polymerase breaks the reporter-quencher proximity and thus allows unquenched emission of fluorescence, which can be detected. An increase in the product targeted by the reporter probe at each PCR cycle therefore causes a proportional increase in fluorescence due to the breakdown of the probe and release of the reporter.

The PCR reaction is prepared as usual (see PCR), and the reporter probe is added. As the reaction commences, during the annealing stage of the PCR both probe and primers anneal to the DNA target. Polymerization of a new DNA strand is initiated from the primers, and once the polymerase reaches the probe, its 5′-3-exonuclease degrades the probe, physically separating the fluorescent reporter from the quencher, resulting in an increase in fluorescence.

Fluorescence is detected and measured in the real-time PCR thermocycler, and its geometric increase corresponding to exponential increase of the product is used to determine the threshold cycle (C_{T}) in each reaction.

Quantitating gene expression by traditional methods presents several problems. Firstly, detection of mRNA on a Northern blot or PCR products on a gel or Southern blot is time-consuming and does not allow precise quantitation. Also, over the 20-40 cycles of a typical PCR reaction, the amount of product reaches a plateau determined more by the amount of primers in the reaction mix than by the input template/sample.

Relative concentrations of DNA present during the exponential phase of the reaction are determined by plotting fluorescence against cycle number on a logarithmic scale (so an exponentially increasing quantity will give a straight line). A threshold for detection of fluorescence above background is determined. The cycle at which the fluorescence from a sample crosses the threshold is called the cycle threshold, Cₜ. Since the quantity of DNA doubles every cycle during the exponential phase, relative amounts of DNA can be calculated, e.g., a sample whose Cₜ is 3 cycles earlier than another's has 2³=8 times more template.

Amounts of RNA or DNA are then determined by comparing the results to a standard curve produced by RT-PCR of serial dilutions (e.g., undiluted, 1:4, 1:16, 1:64) of a known amount of RNA or DNA. As mentioned above, to accurately quantify gene expression, the measured amount of RNA from the gene of interest is divided by the amount of RNA from a housekeeping gene measured in the same sample to normalize for possible variation in the amount and quality of RNA between different samples. This normalization permits accurate comparison of expression of the gene of interest between different samples, provided that the expression of the reference (housekeeping) gene used in the normalization is very similar across all the samples. Choosing a reference gene fulfilling this criterion is therefore of high importance, and often challenging, because only very few genes show equal levels of expression across a range of different conditions or tissues.
iii. Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.
iv. Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al. (1989).

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).
v. Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al. (1989). For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. Using the sequences disclosed herein, oligonucleotide primers may be designed to permit the amplification of sequences that may then be analyzed by direct sequencing.
vi. Kit Components

All the essential materials and reagents required for detecting survivin expression may be assembled together in a kit. This generally will comprise preselected primers and probes. Also included may be enzymes suitable for amplifying nucleic acids including various polymerases (RT, Taq, Sequenase™, etc.), deoxynucleotides and buffers to provide the necessary reaction mixture for amplification. Such kits also generally will comprise, in suitable means, distinct containers for each individual reagent and enzyme as well as for each primer or probe.

vii. Chip Technologies

Specifically contemplated by the present inventors are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Pease et al. (1994); Fodor et al. (1991).

2. Immunodiagnostics

Antibodies of can be used in characterizing the survivin content of target cells through techniques such as ELISAs and Western blotting. The use of antibodies in the present invention in an ELISA assay is contemplated. For example, antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for a tumor suppressor that differs the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° to about 27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

Antibodies can also find use in immunoblots or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the tumor suppressor are considered to be of particular use in this regard.

### V. Combination Cancer Therapies

Any of the following therapies may be combined with miRNA-based therapies to increase the killing of cancer cells, the inhibition of cancer cell growth, the inhibition of metastasis, the inhibition of angiogenesis or otherwise improve the reverse or reduction of malignant phenotype of tumor cells. These compositions would be provided in a combined amount effective to kill or inhibit proliferation of the cell. This process may involve contacting the cells with an miRNA or expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the miRNA or expression construct and the other includes the agent.

Alternatively, the miRNA treatment may precede or follow the other agent treatment by intervals ranging from minutes to weeks. In embodiments where the agents are applied separately to the cell, one would generally ensure that a significant period of time did not expire between the time of each delivery, such that the agents would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either agent will be desired. Various combinations may be employed, where the miRNA therapy is “A” and the other agent is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Again, to achieve cell killing, both agents are delivered to a cell in a combined amount effective to kill the cell.

A. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Intratumoral injection prior to surgery or upon excision of part of all of cancerous cells, tissue, or tumor, a cavity may be formed in the body. Treatment may be accomplished by perfusion, direct injection or local application of these areas with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

B. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.
1. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents, some of which are discussed below.
2. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the syntheisis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.
3. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle.

Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms these compounds are administered through bolus injections intravenously at doses ranging from 25-75 m g/m² at 21 day intervals for adriamycin, to 35-100 mg/m² for etoposide intravenously or orally.
4. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.
5. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.
6. Other Agents

Other agents that may be used include bevacizumab (brand name Avastin®), gefitinib (Iressa®), trastuzumab (Herceptin®), cetuximab (Erbitux®), panitumumab (Vectibix®), bortezomib (Velcade®), and Gleevec. In addition, growth factor inhibitors and small molecule kinase inhibitors have utility in the present invention as well. All therapies described in Cancer: Principles and Practice of Oncology (7^{th} Ed.), 2004, and Clinical Oncology (3^{rd} Ed., 2004) are hereby incorporated by reference. The following additional therapies are encompassed, as well.

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with p53 gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

Tumor Necrosis Factor is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

The use of sex hormones according to the methods described herein in the treatment of cancer. While the methods described herein are not limited to the treatment of a specific cancer, this use of hormones has benefits with respect to cancers of the breast, prostate, and endometrial (lining of the uterus). Examples of these hormones are estrogens, anti-estrogens, progesterones, and androgens.

Corticosteroid hormones are useful in treating some types of cancer (lymphoma, leukemias, and multiple myeloma). Corticosteroid hormones can increase the effectiveness of other chemotherapy agents, and consequently, they are frequently used in combination treatments. Prednisone and dexamethasone are examples of corticosteroid hormones.

D. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques. Stereotactic radiotherapy is used to treat brain tumors. This technique directs the radiotherapy from many different angles so that the dose going to the tumor is very high and the dose affecting surrounding healthy tissue is very low. Before treatment, several scans are analyzed by computers to ensure that the radiotherapy is precisely targeted, and the patient's head is held still in a specially made frame while receiving radiotherapy. Several doses are given.

Stereotactic radio-surgery (gamma knife) for brain and other tumors does not use a knife, but very precisely targeted beams of gamma radiotherapy from hundreds of different angles. Only one session of radiotherapy, taking about four to five hours, is needed. For this treatment you will have a specially made metal frame attached to your head. Then several scans and x-rays are carried out to find the precise area where the treatment is needed. During the radiotherapy for brain tumors, the patient lies with their head in a large helmet, which has hundreds of holes in it to allow the radiotherapy beams through. Related approaches permit positioning for the treatment of tumors in other areas of the body.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

### VI. Examples

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Reagents and antibodies. The miRIDIAN miRNA specific mimics and their negative controls were purchased from Thermo Scientific Dharmacon (Lafayette, Colo.). Paclitaxel (Ben Venue Labs, Inc., Bedford, Ohio) was obtained from University of Colorado Hospital pharmacy.

The primary antibodies used for western blot analyses were obtained as follows: Survivin (clone EPR2675) (Epitomics, Inc., Burlingame, Calif.); MCl-1 (clone 22) (BD Biosciences, San Jose, Calif.); BCl-xL, caspase-3 (8G10), PARP (Cell Signaling Technology, Inc., Beverly, Mass.); β-actin (AC-75) (Sigma). All other reagents were purchased from Sigma unless otherwise specified.

Cells and cell culture. Human breast cancer cell lines BT474, MDA-MB-453, and SKBR3 were obtained from ATCC (Manassas, Va.). The erbB3-transfectants SKBR3.B3.1 and SKBR3.B3.2 were developed from SKBR3 cells and described previously (Wang et al., 2010). BT474-HR20, a trastuzumab-resistant subline, was derived from BT474 cells and maintained well in the presence of 20 μg/ml of trastuzumab (Liu et al., 2011; Howe et al., 2011). The identity of all the cell lines was recently confirmed with DNA profiling by the DNA Sequencing and Analysis Core facility at University of Colorado Cancer Center. All cells were maintained in Dulbecco' s Modified Eagle's Medium: Nutrient Mix F-12 (D-MEM/F-12 1:1) supplemented with 10% fetal bovine serum (FBS). All cell lines were cultured in a 37° C. humidified atmosphere containing 95% air and 5% CO₂ and were split twice a week.

Analysis of miRNA expression. Total RNA, including small RNA, was extracted and purified using the miRNeasy® Mini Kit (QIAGEN Inc., Valencia, Calif.) following the manufacturer's instructions. For miRNA analysis, TaqMan® MicroRNA Reverse Transcription kit (Applied Biosystems) was first used to generate cDNA with the hairpin primers which are specific to the mature miRNA and will not bind to the precursors. The expression levels of miR-203 and miR-542-3p were then measured by quantitative real-time (qRT)-PCR using TaqMan MicroRNA Assays (Assay ID: 000507 and 001284, respectively; Applied Biosystems). RNU6B was used as an internal control to normalize all data using the TaqMan RNU6B Assay (Assay ID: 001093; Applied Biosystems). The relative miRNA levels were calculated using the comparative Ct method (AACt) as reported (Howe et al., 2011).

Transfection of cells with miRNA mimic. Cell transfection with the miRIDIAN miRNA mimics or negative controls was carried out using HiPerFect Transfection Reagent (QIAGEN) according to the manufacturer's protocol.

Quantification of apoptosis. A specific apoptotic ELISA kit (Roche Diagnostics Corp., Indianapolis, Ind.) was used to quantitatively measure cytoplasmic histone-associated DNA fragments (mononucleosomes and oligonucleosomes) as previously reported (Wang et al., 2010; Huang et al., 2010; Huang et al., 2009). This enzyme immunoassay was performed according to the manufacturer's instructions.

Western blot analysis. Protein expression levels were determined by western blot analysis as described previously (Wang et al., 2010; Huang et al., 2009; Huang et al., 2011). Equal amounts of total cell lysates were boiled in Laemmli SDS-sample buffer, resolved by SDS-PAGE, transferred to nitrocellulose membrane (Bio-Rad Laboratories, Hercules, Calif.), and probed with the primary antibodies described in the figure legends. After the blots were incubated with horseradish peroxidase-labeled secondary antibody, the signals were detected using the enhanced chemiluminescence reagents (GE Healthcare Bio-Sciences Corp., Piscataway, N.J.).

Statistical analysis. All experiments were performed at least in duplicate. Statistical analyses of the experimental data were determined using a two sided student's t-test. A P-value <0.05 was deemed statistically significant.

### Example 2

### Results

Ectopic expression of miR-203 and/or miR542-3p specifically down-regulates Survivin in erbB2-driven (erbB2+) breast cancer cells. The inventor and others have shown that the erbB receptors, such as the epidermal growth factor receptor (EGFR), erbB2, and erbB3, are capable of inducing Survivin expression in breast cancer cells (Wang et al., 2010; Asanuma et al., 2005; Xia et al., 2006). To investigate whether miR-203 and miR-542-3p-replacement therapy may be a viable strategy inhibiting Survivin for cancer treatment, the inventor first tested the inhibitory effects of miR-203 and/or miR-542-3p on Survivin in erbB2+ breast cancer cells. The control mimic or mimics of miR-203 and/or miR-542-3p were transiently transfected into SKBR3.B3.1 and SKBR3.B3.2 cells (both are erbB3-transfectant clones derived from SKBR3 cells, and express higher levels of Survivin than SKBR3 cells (Wang et al., 2010)). Quantitative real-time (qRT)-PCR assays were then performed to measure the levels of miRNAs. The data showed that the expression levels of miR-203 and/or miR-542-3p were dramatically increased following mimic(s) transfection. The miRNA mimics used were specific, as each enhanced miRNA expression was only observed in the transfection of their corresponding miRNA mimic (FIG. 1A). Further studies with western blot analyses revealed that the mimic of either miR-203 or miR-542-3p clearly reduced the protein levels of Survivin. It appeared that miR-542-3p had a more profound inhibition on Survivin than miR-203 did. Interestingly, the combination of both miR-203 and miR-542-3p mimics much more effectively down-regulated Survivin than either of the two miRNA mimics in both SKBR3.B3.1 and SKBR3.B3.2 cells (FIG. 1B). The mimic(s) of miR-203 and/or miR-542-3p had no effect on MCl-1 and BCl-xL, which are functionally related to Survivin (Jane et al., 2011).

To further confirm these observations, the inventor performed additional assays in multiple erbB2+ breast cancer cell lines. Similar results were obtained. FIGS. 2A-B that show that while both miR-203 and miR-542-3p mimic were able to down-regulate Survivin, their combinations much more effectively reduced the protein levels of Survivin, but not Mcl-1 and Bcl-xL in MDA-MB-453 and BT474, two well-known erbB2+ breast cancer cell lines, and BT474-HR20 cells (a trastuzumab-resistant subline derived from BT474 cells (Huang et al., 2010)). Taken together, these data suggest that miR-203 and miR-542-3p exhibit functional cooperation to specifically target Survivin in erbB2+ breast cancer cells.

Reactivation of miR-203 and/or miR542-3p with exogenous expression significantly enhances paclitaxel-induced apoptosis in the otherwise resistant breast cancer cells. The inventor's previous data showed that up-regulation of Survivin was required for erbB2/erbB3-mediated anti-apoptotic cell death-induced by paclitaxel, and specific knockdown of Survivin with shRNA overcame this resistance (Wang et al., 2010). Since the inventor observed that ectopic expression of miR-203 and/or miR-542-3p specifically targeted Survivin in SKBR3.B3.1, SKBR3.B3.2, and BT474-HR20 cells (FIGS. 1B and 2B), it is conceivable that increased miR-203 and/or miR-542-3p should be able to abrogate paclitaxel resistance and enhance paclitaxel-induced apoptosis in the resistant cells. The control mimic or mimics of miR-203 and/or miR-542-3p were transiently transfected into SKBR3.B3.1, SKBR3.B3.2, or BT474-HR20 cells. After 24 hrs, the cells were then untreated or treated with paclitaxel for another 24 hrs. Western blot analyses showed that when compared with paclitaxel treatment alone in control mimic transfection, the mimic of either miR-203 or miR-542-3p clearly enhanced paclitaxel-induced PARP cleavage, a hall marker of apoptosis, and caspase-3 activation (cleaved caspase-3) in both SKBR3.B3.1 and SKBR3.B3.2 cells (FIG. 3A). Consistent with the inhibitory effects on Survivin, miR-542-3p displayed more profound effects than miR-203, and the combinations of two miRNA mimics exerted a much more potent activity than one mimic to enhance paclitaxel-induced PARP cleavage and caspase-3 activation. Furthermore, a specific apoptotic-ELISA revealed that as compared to single miRNA, simultaneous increase of both miR-203 and miR-542-3p more effectively enhanced paclitaxel-induced apoptosis in both SKBR3.B3.1 and SKBR3.B3.2 cells (FIG. 3B). To strengthen the hypothesis, the inventor performed the same studies and obtained similar results in another breast cancer cell line, BT474-HR20 (FIGS. 3C-D), which acquired paclitaxel-resistant phenotypes via a different mechanism. BT474-HR20 cells were derived from BT474 cells through long-term exposure to transtuzumab in cell culture (Huang et al., 2010). They also express higher levels of Survivin and are more resistant to paclitaxel treatment than the parental BT474 cells (data not shown). Collectively, these findings strongly indicate that reactivation of miR-203 and/or miR542-3p with exogenous expression is capable of overcoming resistance, and significantly enhances paclitaxel-induced apoptosis in the otherwise resistant breast cancer cells.

### Example 3

### Discussion

Survivin is usually expressed in embryonic tissues during development, but undetectable in most normal adult tissues. Numerous studies show that Survivin is selectively expressed in almost all types of human malignancies and its overexpression positively correlates with poor clinical outcome (Liu et al., 2008; Mita et al., 2008). This differential expression of Survivin in tumors and normal tissues draws a great interest to identify strategies targeting Survivin for cancer therapy (Mita et al., 2008; Altieri, 2008). Currently, there is no reported study considering epigenetic approach inhibiting Survivin to treat human cancers.

Here, the inventor demonstrates that increasing expression of miR-203 and/or miR542-3p is a viable strategy targeting Survivin to overcome paclitaxel resistance and re-sensitize the resistant cells to paclitaxel-induced apoptosis. Although these data were generated from in vitro cell culture studies, they provide compelling evidence suggesting that reactivation of miR-203 and/or miR542-3p via exogenous expression might be effective to inhibit Survivin in vivo as well. This notion is strongly supported by a recent report indicating that a novel approach—polyethylenimine (PEI)-mediated delivery of unmodified miRNA is an efficient strategy being tested as a miRNA replacement therapy in an animal model of colon cancer (Ibrahim et al., 2011). Thus, the inventor will test the hypothesis in animal experiments using tumor xenografts model established from BT474-HR20 cells in nude mice, hopefully confirming in vitro data (FIGS. 2B and 3C-D). The inventor will use the low molecular weight branched PEI (Hobel et al., 2010) as a platform to deliver either miR-203, or miR-542-3p, or their combination into the mice to determine the role of miR-203 and/or miR542-3p-replacement therapy in enhancing paclitaxel-mediated apoptosis in vivo.

These studies focus on enhancing paclitaxel sensitivity against erbB2+ breast cancer. In fact, paclitaxel is widely used to treat prostate, colorectal, ovarian, and other cancers. In addition to paclitaxel, overexpression of Survivin also leads to therapeutic resistance to other chemotherapeutic drugs, such as docetaxel, doxorubicin, carboplatin, and etc. in cancer treatment. Therefore, targeting Survivin with miR-203 and/or miR542-3p-replacement therapy may also be able to enhance the efficacy of other chemotherapeutic drugs against a wide variety of human cancers, thus have a much broader impact on cancer treatment. Further, studies using both in vitro cell culture system and in vivo animal models are warranted to elucidate this hypothesis.

In summary, the inventor has demonstrated that elevated expression of miR-203 and/or miR542-3p specifically down-regulates Survivin, and significantly enhances paclitaxel-induced apoptosis in the otherwise resistant, erbB2+ breast cancer cells. These findings, together with previous reports on the role of Survivin in erbB2/erbB3-mediated paclitaxel resistance, provide a strong rationale to develop a miR-203/miR542-3p-replacement therapy targeting Survivin. Such strategy should significantly enhance the anti-tumor activity of chemotherapies for cancer treatment.

All of the compositions and/or methods disclosed and claimed herein can be made and executed without undue experimentation in light of the present disclosure. While the compositions and methods of this invention have been described in terms of particular embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VII. References

The following references, to the extent that they provide exemplary procedural or other details supplementary to those set forth herein, are specifically incorporated herein by reference:
U.S. Pat. No. 4,683,195
U.S. Pat. No. 4,683,202
U.S. Pat. No. 4,800,159
U.S. Pat. No. 4,883,750
U.S. Pat. No. 5,279,721
U.S. Patent Publn. 2007/0213292
U.S. Patent Publn. 2007/0287831,
U.S. Patent Publn. 2008/0032945
Adams et al., Semin. Reprod. Med., 26(6):522-536, 2008.
Altieri, Nat. Rev. Cancer, 8(1):61-70, 2008.
Ambros et al. RNA, 9:277-279, 2003.
Arora et al., Sci. Transl. Med., 4(133):133ra156, 2012.
Asanuma et al., Cancer Res., 65(23):11018-11025, 2005.
Bartel, Cell, 136(2):215-233, 2009.
Bellus, J. Macromol. Sci. Pure Appl. Chem., A31(1): 1355-1376, 1994.
Bray et al., Cancer Lett., 303(1):56-64, 2011.
Brennecke et al., Cell, 113(1):25-36, 2003.
Calin et al., Proc. Natl. Acad. Sci. USA, 99(24):15524-15529, 2002.
Cancer: Principles and Practice of Oncology (7^{th} Ed.), 2004
Care et al. Nat. Med., 13(5):613-618, 2007.
Chang et al., J. Exp. Med., 200:1359-1370, 2004.
Chen et al., Gynecol. Oncol., 94:630-635, 2004.
Clinical Oncology, 3^{rd} Ed., 2004.
Craig et al., Cancer Res., 71(10):3616-3624, 2011.
EPO No. 329 822
EPO No. 320-308
Esquela-Kerscher and Slack, Nat. Rev. Cancer, 6(4):259-269, 2006.
Fatkin et al., J. Clin. Invest., 106(11):1351-1359, 2000.
Fodor et al., Biochemistry, 30(33):8102-8108, 1991.
Formosa et al., Oncogene, 1014, 2012.
Freifelder, In: Physical Biochemistry Applications to Biochemistry and Molecular Biology, 2nd Ed. Wm. Freeman and Co., NY, 1982.
Frohman, In: PCR Protocols. A Guide to Methods and Applications, Academic Press, N.Y., 1990
Furuta et al., Carcinogenesis, 31(5):766-776, 2010.
GB Appln. No. 2 202 328
Hacia et al., Nature Genet., 14:441-449, 1996.
Hobel et al., J. Gene Med., 12(3):287-300, 2010.
Howe et al., Breast Cancer Res., 13(2):R45, 2011.
Huang et al., Cancer Lett., 307(1):72-79, 2011.
Huang et al., Cancer Res., 69(21):8403-8411, 2009.
Huang et al., Cancer Res., 70(3):1204-1214, 2010.
Hutvagner et al., PLoS Biol., 2(4):E98, 2004.
Ibrahim et al., Cancer Res., 71(15):5214-5224, 2011.
Innis, et al., In: PCR Protocols. A guide to Methods and Application, Academic Press, Inc. San Diego, 1990.
Jane et al., Mol. Cancer Ther., 10(1):198-208, 2011.
Kelly et al., Mol. Cancer, 10:35, 2011.
Kiriazis and Kranias, Annu. Rev. Physiol., 62:321-351, 2000.
Krenz and Robbins, J. Am. Coll. Cardiol., 44:2390-2397, 2004.
Kwoh et al., Proc. Natl. Acad. Sci. USA, 86(4):1173-7, 1989.
Lagos-Quintana et al., Science, 294(5543):853-858, 2001.
Lau et al., Science, 294(5543):858-862, 2001.
Lee and Ambros, Science, 294(5543):862-864, 2001.
Liu et al., Cancer Biol. Ther., 7(7):1053-1060, 2008.
Liu et al., Cell Cycle, 10(17):2959-2966, 2011.
Lowes et al., J. Clin. Invest., 100(9):2315-2324, 1997.
Meister et al. Mol. Cell, 15:185-197, 2004.
Mita et al., Clin Cancer Res., 14(16):5000-5005, 2008.
Nelson and Weiss, Mol. Cancer Ther., 7(12):3655-3660, 2008.
Ohara et al., Proc. Natl. Acad. Sci. USA, 86:5673-5677, 1989.
Oneyama et al., “MicroRNA-mediated upregulation of integrin-linked kinase promotes Src-induced tumor progression,” Oncogene 31: 1623-1635, 2012.
Pasquinelli, Ann. Rev. Cell Dev. Biol., 18:495-513, 2002.
PCT Appln. PCT/US87/00880
PCT Appln. PCT/US89/01025
PCT Appln. WO 88/10315
PCT Appln. WO 89/06700
PCT Appln. WO 90/07641
Pease et al., Proc. Natl. Acad. Sci. USA, 91:5022-5026, 1994.
Pignon et al., Hum. Mutat., 3: 126-132, 1994.
Saini et al., Clin. Cancer Res., 17(16):5287-5298, 2011.
Sambrook et al., In: Molecular cloning: a laboratory manual, 2^{nd} Ed., Cold Spring Harbor Laboratory Press, Cold Spring Harbor, N.Y., 1989.
Shoemaker et al., Nature Genetics, 14:450-456, 1996.
Summerton and Weller, Antisense Nucleic Acid Drug Dev., 7(3):187-95, 1997.
Walker et al., Nucleic Acids Res. 20(7):1691-1696, 1992.
Wang et al., Oncogene, 29(29):4225-4236, 2010.
Winter et al., Nat. Cell Biol., 11(3):228-234, 2009.
Wu et al., Genomics, 4:560-569, 1989.
Xia et al., Cancer Res., 66(3):1640-1647, 2006.
Xu et al., Curr. Biol., 13(9):790-795, 2003.
Yang et al., Biomacromolecules, 8:3422-3428, 2007.
Yoon et al., “Induction of growth arrest by miR-542-3p that targets surviving,” FEBS Letters, 584: 4048-4052, 2010.
Zeng et al., Cancer Res., 62(13):3630-3635, 2002.
Zhang et al., Dev. Biol., 302(1):1-12, 2007.
Zhang et al., Genes Cancer, 2(8):782-791, 2011.

1. A method of inhibiting a survivin-positive cancer cell comprising contacting said cell with miR-203 and/or miR-542-3p in combination with an anti-cancer chemotherapeutic agent.

2. The method of claim 1, wherein said cancer cell is contacted with miR-203 and not miR-542-3p.

3. The method of claim 1, wherein said cancer cell is contacted with miR-542-3p and not miR-203.

4. The method of claim 1, wherein said cancer cell is contacted with miR-203 and miR-542-3p.

5. The method of claim 1, wherein said anti-cancer chemotherapeutic agent is an anti-mitotic agent, alkylating agent, and cytotoxic antibiotic.

6-8. (canceled)

9. The method of claim 1, wherein said cancer cell is a breast cancer cell, prostate cancer cell, ovarian cancer cell, lung cancer cell, gastric cancer cell, esophageal cancer cell, and pancreatic cancer cell.

10. The method of claim 1, wherein said cancer cell is a recurrent cancer cell, a metastatic cancer cell or a multi-drug resistant cancer cell.

11-13. (canceled)

14. The method of claim 1, wherein inhibiting comprises inhibiting growth of said cancer cell or killing said cancer cell.

15. (canceled)

16. The method of claim 1, further comprising contacting said cancer cell with an additional anti-cancer therapy.

17. (canceled)

18. A method of treating a subject exhibiting a survivin-positive cancer comprising administering to said subject miR-203 and/or miR-542-3p in combination with an anti-cancer chemotherapeutic agent.

19. The method of claim 18, wherein said subject is administered miR-203 and not miR-542-3p.

20. The method of claim 18, wherein said subject is administered miR-542-3p and not miR-203.

21. The method of claim 18, wherein said subject is administered miR-203 and miR-542-3p.

22. The method of claim 18, wherein said anti-cancer chemotherapeutic agent is an anti-mitotic agent, alkylating agent, and cytotoxic antibiotic.

23. (canceled)

24. The method of claim 18, wherein said cancer is breast cancer, prostate cancer, ovarian cancer, lung cancer, gastric cancer, esophageal cancer, and pancreatic cancer.

25. The method of claim 18, wherein said cancer is a recurrent cancer, a metastatic cancer or a multi-drug resistant cancer.

26-28. (canceled)

29. The method of claim 18, wherein inhibiting comprises slowing tumor growth.

30. The method of claim 18, wherein inhibiting comprises delaying tumor metastasis.

31. The method of claim 18, wherein inhibiting comprises killing one or more cells of said cancer.

32. The method of claim 18, further comprising treating said subject with an additional anti-cancer therapy.

33-36. (canceled)
